(19) United States
(12) Patent Application Publication
(10) Pub. No.: US 2005/0038116 A1
(43) Pub. Date: Feb. 17, 2005

(54) Novel class of compounds (Choline derivatives, esp. Stearyl Choline Cloride and other salts) for the treatment of Alzheimer's disease, Down syndrome and central (and/or peripheral) nervous system and memory related disorders or for enhancements

(76) Inventor: Hasmukh B. Patel, Edison, NJ (US)

Correspondence Address: HASMUKH B. PATEL, 44 HANA ROAD, EDISON, NJ, 08817, US

(21) Appl. No.: 10/642,455

(22) Filed: Aug. 15, 2003

(51) Int. Cl. A61K 31/23

(52) U.S. Cl. 514/552

(57) ABSTRACT

This invention is for the treatment of Alzheimer's disease, Down syndrome, Central (or Peripheral) Nervous Systems' disorders or for the enhancement of memory and central nervous system or peripheral nervous system. In this invention a novel class of Choline derivatives are recommended for the above mentioned indications. These derivatives are the esters of Choline. An example of this class was newly synthesized using Stearic acid. This derivative named Stearyl Choline Cloride or Choline Staearate Chloride or Stearic acid ester of Choline was synthesized and tested in animals. The results are positive.

### BACKGROUND OF THE INVENTION

The invention is centered on the use of the Choline derivatives (esters) which are sufficiently hydrophobic to cross the blood brain barrier. The ester moiety can be any chemical group. Thus these esters can be used for the following purposes: The use of the class of compounds described in claim 1 for the treatment of the following:
Alzheimer's disease
Down syndrome
Central Nervous System (CNS) disorders
Peripheral Nervous System (PNS) disorders
Memory related disorders
Enhancements of memory and related function
Enhancement of CNS functions
Enhancements of PNS functions
Improvement of cognition
Improvement in learning and behavior

This invention is for the treatment of Alzheimer's disease, Down syndrome, Central (or Peripheral) Nervous Systems' disorders or for the enhancement of memory and central nervous system or peripheral nervous system. In this invention a novel class of Choline derivatives (See Table 1 and Table 2) are recommended for the above mentioned indications. These derivatives are the esters of Choline. An example of this class was newly synthesized using Stearic acid. This derivative named Stearyl Choline Cloride or Choline Staearate Chloride or Stearic acid ester of Choline was synthesized and tested in animals.

Use of the these Choline ester derivatives will allow the followings:

1. Will help treat Alzheimer's disease.
2. Will help improve memory functions and Central nervous System and/or Peripheral Nervous System disorders.
3. Will help the treatment of Down Syndrome.

### BRIEF SUMMARY OF THE INVENTION

The current invention is centered on the use synthesis and use of Choline esters (see Table 1) (esp. Stearyl Choline Salts) for the treatment of the following indications:
a. Alzheimer's disease
b. Down syndrome
c. Central Nervous System (CNS) disorders
d. Peripheral Nervous System (PNS) disorders
e. Memory related disorders
f. Enhancements of memory and related function
g. Enhancement of CNS functions
h. Enhancements of PNS functions
i. Improvement of cognition
j. Improvement in learning and behavior

Following are the claimed benefits of the use Choline esters (esp. Stearyl Choline Salt):
1. Better absorption through the GI system
2. Better penetration through the blood brain barrier.
3. No toxicity as it is made up of natural compounds
4. No side effects

### DETAILED DESCRIPTION OF THE INVENTION

This invention is for the treatment of Alzheimer's disease, Down syndrome, Central (or Peripheral) Nervous Systems' disorders or for the enhancement of memory and central nervous system or peripheral nervous system. In this invention a novel class of Choline derivatives are recommended for the above mentioned indications. These derivatives are the esters of Choline. An example of this class was newly synthesized using Stearic acid. This derivative named Stearyl Choline Cloride or Choline Staearate Chloride or Stearic acid ester of Choline was synthesized and tested in animals.

Use of the these Choline ester derivatives will allow the followings:

Will help treat Alzheimer's disease.
Will help improve memory functions and
Will help the treatment of Down Syndrome.
In treatment of Central Nervous System (CNS) disorders
In treatment of Peripheral Nervous System (PNS) disorders
Help in treating memory related disorders
Will help in enhancements of memory and related function
Will help in enhancement of CNS functions
Will help in enhancements of PNS functions
Will help in improvement of cognition
Will help in improvement in learning and behavior
   Other benefits of the use of Choline esters are:
Better absorption through the GI system
Better penetration through the blood brain barrier.
No toxicity as it is made up of natural compounds

No side effects

TABLE 1
Structures of Choline Esters

| | |
|---|---|
| 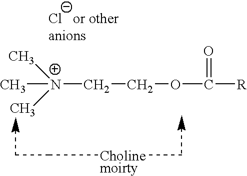 | |
| R = —(CH₂)₁₇—CH₃ = | stearyl choline |
| | chloride or choine |
| | stearate chloride |
| R = | Any other chemical |
| | moiety |

[image]

1. Invention of Choline ester derivatives including Stearyl Choline Chloride (other names Choline Stearate Chloride, Choline ester of Stearic acid) are claimed for the treatment of the indications listed in claim 3.

2. Stearyl Choline Salts or Choline Stearate Chloride or other salts is claimed as novel compounds for the treatments claimed in claim 3.

3. The use of the class of compounds described in claim 1 and 2 for the treatment of the following:
Alzheimer's disease
Down syndrome
Central Nervous System (CNS) disorders
Peripheral Nervous System (PNS) disorders
Memory-related disorders
Enhancements of memory and related function
Enhancement of CNS functions
Enhancements of PNS functions
Improvement of cognition
Improvement in learning and behavior
